(19) United States
(12) Patent Application Publication
(10) Pub. No.: US 2026/0076936 A1
(43) Pub. Date: Mar. 19, 2026

(54) SUSTAINED-RELEASE PHARMACEUTICAL COMPOSITION OF SIVELESTAT OR SALT THEREOF AND PREPARATION METHOD THEREOF

(71) Applicant: Beijing Tide Pharmaceutical Co., Ltd., Beijing (CN)

(72) Inventors: Yuhang SUN, Beijing (CN); Zhibing ZHANG, Beijing (CN); Deyue FENG, Beijing (CN); Yunna DING, Beijing (CN); Yajing FAN, Beijing (CN); Yun WU, Beijing (CN); Leling GAN, Beijing (CN)

(73) Assignee: Beijing Tide Pharmaceutical Co., Ltd., Beijing (CN)

(21) Appl. No.: 19/326,926

(22) Filed: Sep. 12, 2025

(30) Foreign Application Priority Data
Sep. 13, 2024 (CN) 202411281700.2
Sep. 13, 2024 (CN) 202411281751.5

(51) Int. Cl. A61K 31/24; A61K 9/00; A61K 9/16

(52) U.S. Cl. CPC A61K 31/24; A61K 9/0075; A61K 9/1617; A61K 9/1623; A61K 9/1635; A61K 9/167; A61K 9/1682

(57) ABSTRACT

The present disclosure relates to the field of pharmaceutics and particularly provides a sustained-release pharmaceutical composition of sivelestat and a preparation method therefor. The pharmaceutical composition included sivelestat and a phospholipid. The present disclosure uses sivelestat, which is poorly soluble in water, as an active ingredient and the phospholipid as a stabilizer. Particles of sivelestat have excellent aerodynamic performance, good stability, a small amount of auxiliary materials, and a simple preparation process. After inhalation, the drug stays in the lungs for a long time and has a good sustained-release effect and a good industrial production prospect.

### CROSS REFERENCE TO RELATED APPLICATIONS

This application claims the benefit of priority from Chinese Patent Application No. 202411281751.5, filed Sep. 13, 2024 and Chinese Patent Application No. 202411281700.2, filed Sep. 13, 2024, the contents of each of which are incorporated herein by reference.

### TECHNICAL FIELD

The present disclosure relates to the field of pharmaceutical formulations and particularly to a pharmaceutical composition of sivelestat or a salt thereof and a preparation method therefor.

### BACKGROUND

Sivelestat sodium can inhibit the activity of neutrophil elastase and is an effective drug for treating acute lung injury (ALI) or acute respiratory distress syndrome (ARDS) accompanied by systemic inflammatory response syndrome. Clinical studies have shown that sivelestat sodium can effectively reduce the lung injury scores of ARDS patients, improve pulmonary function, shorten the duration of mechanical ventilation and intensive care unit stays, and reduce mortality.

Sivelestat sodium was developed by Ono Pharmaceutical Co., Ltd., Japan, and was approved in Japan in April 2002 in the dosage form of a freeze-dried powder for injection with a strength of 100 mg. For administration, a 24-h dose (4.8 mg/kg patient body weight) is diluted in 250-500 mL of normal saline and continuously and intravenously administered for 24 h (equivalent to 0.2 mg·kg⁻¹·h⁻¹); alternatively, a 24-h dose of sivelestat sodium (4.8 mg/kg) is dissolved in normal saline, and the drug solution is drawn into a 50 mL syringe, brought to a total volume of 48 mL, and administered via an intravenous drip micro-infusion pump at a constant rate of 2 mL/h over 24 h; the daily dose can also be divided into 3 parts and continuously administered by intravenous drip infusion, and the maximum duration of treatment is 14 days.

Current studies on sivelestat sodium focus primarily on systemic administration. However, due to the low local drug concentrations during systemic administration, higher doses are administered, which enhances the toxic and side effects of the drug and elevates the financial burden on patients. The lungs, which are relatively rich in blood vessels and have a large absorption area, allow for rapid absorption of inhaled drugs into the bloodstream, achieving an onset of action similar to that of intravenous administration. In addition, inhalation administration enables drugs to reach terminal bronchi and alveoli, resulting in fewer side effects compared to systemic administration.

The aqueous solution of sivelestat sodium has poor stability as the drug is prone to hydrolysis; therefore, it is formulated in the form of a powder for injection. The production of the powder for injection involves a lengthy freeze-drying process, which is both time-consuming and costly. Moreover, the powder for injection has no sustained-release effect and requires continuous 24-h intravenous infusion, leading to poor patient compliance.

CN117982414A, CN114681435A, CN107913261A, and CN104107172A all disclose inhaled formulations of sivelestat sodium. These formulations all use sivelestat sodium as the drug substance, and their formulas contain no sustained-release materials. Consequently, the drug is easily metabolized and cleared, failing to achieve a long-acting, sustained-release effect in the lungs. To achieve the same efficacy as continuous intravenous injection, continuous inhalation administration would be theoretically required, which can cause inconvenience in clinical use.

CN116115589A discloses an inhaled pharmaceutical composition of sivelestat sodium and a preparation method therefor. This composition uses distearoylphosphatidylethanolamine-polyethylene glycol, which has a sustained-release function, as a key auxiliary material to address the problem that the drug has low solubility and poor stability. However, it does not mention any sustained-release effect. Moreover, the composition uses a large amount of the sustained-release material, which is 10-40 times that of the drug. The preparation process is relatively complex: the components need to be dissolved separately, then mixed, and then evaporated under reduced pressure to form a drug-containing lipid film; then, water is added for reconstitution, and mannitol is added as an excipient; finally, the mixture is freeze-dried.

### SUMMARY OF THE INVENTION

To address the technical problems described above, the present disclosure aims to provide sustained-release particles of sivelestat that use sivelestat, which is poorly soluble in water, as an active ingredient and a phospholipid as a stabilizer. The particles have excellent aerodynamic performance, good stability, and a simple preparation process. After inhalation, the drug stays in the lungs for a long time and has a good sustained-release effect and a good industrial production prospect.

Unlike sivelestat sodium, sivelestat has low solubility, good stability, and a slow dissolution rate in water. After the particles are coated with a layer of the phospholipid, the release of the drug can be further delayed, and the biocompatibility is also improved. After atomization and inhalation, the particles stay in the lungs for a long time and can slowly release the drug.

The present disclosure provides a sustained-release sivelestat-containing pharmaceutical composition comprising sivelestat and a phospholipid.

In the present disclosure, as one of the embodiments, sivelestat and the phospholipid form a solid dispersion.

In the present disclosure, as one of the embodiments, a mass ratio of sivelestat to the phospholipid is 1:1 to 1:6, preferably 1:3 to 1:5.

Illustratively, the mass ratio may be 1:1, 1:2, 1:3, 1:4, 1:5, or 1:6.

In the present disclosure, as one of the embodiments, the pharmaceutical composition comprises sivelestat, the phospholipid, and an aqueous solution and does not comprise an organic solvent, wherein the aqueous solution is selected from water for injection, normal saline, and aqueous glucose.

In the present disclosure, as one of the embodiments, in the composition comprising the aqueous solution described above, sivelestat accounts for 0.05-5%, preferably 0.1-3.0%, by mass of the composition, and the phospholipid accounts for 0.15-1.5%, preferably 0.3-1.0%, by mass of the composition.

Illustratively, sivelestat may account for 0.05%, 0.07%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.2%, 3.4%, 3.6%, 3.8%, 4.0%, 4.1%, 4.3%, 4.4%, 4.5%, 4.7%, 4.8%, 4.9%, or 5.0%, and the phospholipid may account for 0.15%, 0.17%, 0.19%, 0.2%, 0.21%, 0.22%, 0.23%, 0.25%, 0.26%, 0.27%, 0.28%, 0.3%, 0.4%, 0.5%, 0.6%, 0.7%, 0.8%, 0.9%, or 1.0%.

In the present disclosure, as one of the embodiments, the pharmaceutical composition comprises sivelestat, the phospholipid, and a drying protectant and does not comprise an organic solvent.

In the present disclosure, as one of the embodiments, in the composition comprising the drying protectant, sivelestat accounts for 5-40%, preferably 10-30%, of the amount of the composition.

Illustratively, sivelestat may account for 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In the present disclosure, as one of the embodiments, in the composition, the phospholipid accounts for 2.5-50%, preferably 5-42%, more preferably 10-37.5%, and further preferably 15-35%, by mass of the composition.

Illustratively, the phospholipid may account for 2.5%, 2.6%, 2.7%, 2.9%, 3%, 3.5%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

In the present disclosure, as one of the embodiments, the drying protectant accounts for 25-75%, preferably 40-60%.

Illustratively, the drying protectant may account for 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%.

In the present disclosure, as one of the embodiments, the phospholipid is selected from one or a combination of two or more of egg yolk lecithin, soybean phospholipid, and a synthetic phospholipid; preferably, the phospholipid is egg yolk lecithin.

In the present disclosure, as one of the embodiments, the drying protectant is one of or a combination of two or more of fructose, xylitol, maltitol, erythritol, povidone, polyethylene glycol, hydroxypropyl methylcellulose, hydroxypropyl cellulose, poloxamer, lactose, glucose, sucrose, trehalose, mannitol, and sorbitol, preferably one of or a combination of two or more of lactose, glucose, sucrose, trehalose, mannitol, and sorbitol, and further preferably mannitol.

In the present disclosure, as one of the embodiments, the pharmaceutical composition comprises:

| Composition | Formula 1-1 (parts by weight) |
|---|---|
| Sivelestat | 1.0 |
| Egg yolk lecithin | 0.5 and |
| Mannitol | 1.0 |

| Composition | Formula 2-1 (parts by weight) |
|---|---|
| Sivelestat | 1.0 |
| Egg yolk lecithin | 1.0 and |
| Mannitol | 1.0 |

| Composition | Formula 3-1 (parts by weight) |
|---|---|
| Sivelestat | 1.0 |
| Egg yolk lecithin | 2.0 and |
| Mannitol | 1.0 |

| Composition | Formula 4-1 (parts by weight) |
|---|---|
| Sivelestat | 1.0 |
| Egg yolk lecithin | 3.0 and |
| Mannitol | 2.0 |

| Composition | Formula 5-1 (parts by weight) |
|---|---|
| Sivelestat | 1.0 |
| Egg yolk lecithin | 3.0 and |
| Mannitol | 4.0 |

| Composition | Formula 6-1 (parts by weight) |
|---|---|
| Sivelestat | 1.0 |
| Egg yolk lecithin | 3.0 and |
| Mannitol | 6.0 |

| Composition | Formula 7-1 (parts by weight) |
|---|---|
| Sivelestat | 1.0 |
| Egg yolk lecithin | 5.0 and |
| Mannitol | 6.0 |

| Composition | Formula 8-1 (parts by weight) |
|---|---|
| Sivelestat | 1.0 |
| Egg yolk lecithin | 5.0 and |
| Mannitol | 18.0 |

| Composition | Formula 9-1 (parts by weight) |
|---|---|
| Sivelestat | 1.0 |
| Egg yolk lecithin | 0.25 and |
| Mannitol | 8.75 |

| Composition | Formula 10-1 (parts by weight) |
|---|---|
| Sivelestat | 0.1 |
| Egg yolk lecithin | 0.3 and |
| Normal saline | 199.6 |

| Composition | Formula 11-1 (parts by weight) |
|---|---|
| Sivelestat | 10 |
| Egg yolk lecithin | 3.0 and |
| Normal saline | 187.0 |

In the present disclosure, as one of the embodiments, a dosage form of the pharmaceutical composition is a freeze-dried powder for injection, a powder for inhalation (dry powder inhalant), an aerosol, or a suspension.

The present disclosure provides a preparation method for a sivelestat-containing pharmaceutical composition, and the method comprises the following steps:
(1) directly grinding sivelestat and the phospholipid to obtain a drug-phospholipid solid dispersion; and
(2) adding water for injection to the solid dispersion, and mixing well (preferably homogenizing under increased pressure) to obtain a suspension.

In the present disclosure, as one of the embodiments, the method comprises the following step:
(3) spray-drying the suspension obtained in step (2) or adding a drying protectant to the suspension, mixing well, and then spray-drying to obtain dry powder particles.

In the present disclosure, as one of the embodiments, the method comprises the following in step (2): the homogenizing under increased pressure refers to performing homogenization 1-10 times under a pressure of 200-1000 bar; preferably, homogenization is performed 6 times under a pressure of 700 bar.

In the present disclosure, as one of the embodiments, the method comprises the following in step (3): spray drying is performed at a temperature of 90-110° C.

In the present disclosure, as one of the embodiments, the dry powder particles may be further mixed with a pharmaceutically acceptable auxiliary material to prepare a powder for inhalation (inhalable dry powder) or an aerosol.

The present disclosure further provides a reconstituted formulation comprising the dry powder particles prepared by the present disclosure and water for injection, normal saline for injection, or aqueous glucose for injection.

The present disclosure further provides use of a sustained-release sivelestat-containing pharmaceutical composition or a reconstituted formulation thereof or a formulation prepared by the method of the present disclosure in the preparation of a medicament for treating an inflammatory response, bronchiectasis, acute lung injury, or acute respiratory distress syndrome.

In the present disclosure, as one of the embodiments, in the pharmaceutical composition, sivelestat or the salt thereof and the phospholipid form phospholipid particles loaded with sivelestat or the salt thereof.

In the present disclosure, as one of the embodiments, in the pharmaceutical composition, a mass ratio of the phospholipid to sivelestat or the salt thereof is 1:1 to 10:1, as one of the embodiments, preferably 2:1 to 8:1, and as one of the embodiments, further preferably 3:1 to 6:1, based on the mass of the pharmaceutical composition.

Illustratively, the mass ratio may be 2:1, 3:1, 4:1, 5:1, 6:1, 7:1, 8:1, 9:1, or 10:1.

In the present disclosure, as one of the embodiments, the phospholipid accounts for 10-60%, preferably 20-50%, and further preferably 20-40%, such as 20-35%, 20-30%, or 30-40%, by mass of the pharmaceutical composition.

Illustratively, the phospholipid may account for 10%, 11%, 13%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

In the present disclosure, as one of the embodiments, the phospholipid is selected from egg yolk lecithin, soybean phospholipid, a synthetic phospholipid, and a combination of two or more thereof.

In the present disclosure, as one of the embodiments, the phospholipid is selected from one or more of a natural phospholipid and a synthetic phospholipid. As one of the embodiments, the natural phospholipid is egg yolk lecithin or soybean phospholipid, and the synthetic phospholipid is, for example, dioleoylphosphatidylcholine.

Preferably, the phospholipid is egg yolk lecithin or soybean phospholipid. Further preferably, the phospholipid is egg yolk lecithin.

In the present disclosure, as one of the embodiments, in the pharmaceutical composition, sivelestat or the salt thereof accounts for 1-50%, preferably 5-40%, more preferably 10-30%, and further preferably 10-25%, by mass of the pharmaceutical composition.

Illustratively, sivelestat or the salt thereof may account for 1%, 2%, 3%, 4%, 5%, 6%, 7%, 8%, 9%, 10%, 11%, 13%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

In the present disclosure, as one of the embodiments, the salt of sivelestat includes, but is not limited to, sivelestat sodium.

In the present disclosure, as one of the embodiments, the pharmaceutical composition further comprises a filler, and the filler accounts for 15-90%, preferably 15-85%, more preferably 35-80%, further preferably 35-75%, and more preferably 50-70%, by mass of the pharmaceutical composition.

Illustratively, the filler may account for 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80%.

In the present disclosure, as one of the embodiments, the filler includes sorbitol, mannitol, lactose, sucrose, glucose, an amino acid, povidone, copovidone, poloxamer, or polyethylene glycol, or a combination of two or more thereof, as one of the embodiments, preferably, the filler is sorbitol, mannitol, lactose, an amino acid, or a combination of two or more thereof.

In the present disclosure, as one of the embodiments, the pharmaceutical composition has a pH of 3.0-5.5.

Illustratively, the pH may be 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In the present disclosure, as one of the embodiments, an adjuster for the pH includes hydrochloric acid, citric acid, acetic acid, or an aqueous solution thereof, as one of the embodiments, the hydrochloric acid solution is a 5% dilute hydrochloric acid solution.

In the present disclosure, as one of the embodiments, the composition comprises:

| Composition | Formula 1-2 (parts by weight) |
|---|---|
| Sivelestat | 2.0 |
| Egg yolk lecithin | 4.0, and |
| Sorbitol | 1.0; |

| Composition | Formula 2-2 (parts by weight) |
|---|---|
| Sivelestat | 2.0 |
| Egg yolk lecithin | 4.0, and |
| Sorbitol | 4.0; |

| Composition | Formula 3-2 (parts by weight) |
|---|---|
| Sivelestat | 2.0 |
| Egg yolk lecithin | 4.0, and |
| Povidone K30 | 8.0; |

| Composition | Formula 4-2 (parts by weight) |
|---|---|
| Sivelestat | 2.0 |
| Soybean phospholipid | 4.0, and |
| Povidone K30 | 8.0; |

| Composition | Formula 5-2 (parts by weight) |
|---|---|
| Sivelestat | 2.0 |
| Dioleoylphosphatidylcholine | 4.0, and |
| Povidone K30 | 8.0; |

| Composition | Formula 6-2 (parts by weight) |
|---|---|
| Sivelestat sodium | 2.0 |
| Egg yolk lecithin | 0.5, and |
| Povidone K30 | 4.0; |

| Composition | Formula 7-2 (parts by weight) |
|---|---|
| Sivelestat sodium | 2.0 |
| Egg yolk lecithin | 2.0, and |
| Povidone K30 | 4.0; |

| Composition | Formula 8-2 (parts by weight) |
|---|---|
| Sivelestat sodium | 2.0 |
| Egg yolk lecithin | 6.0, and |
| Povidone K30 | 12; |

| Composition | Formula 9-2 (parts by weight) |
|---|---|
| Sivelestat sodium | 1.0 |
| Egg yolk lecithin | 5.0, and |
| Povidone K30 | 10. |

In the present disclosure, as one of the embodiments, the composition is further prepared into dry powder particles.

In the present disclosure, as one of the embodiments, the dry powder particles described above are further mixed with a pharmaceutically acceptable auxiliary material to prepare a pharmaceutically acceptable formulation, such as a powder for inhalation (dry powder inhalant), an aerosol, a freeze-dried powder for injection, or a suspension.

In the present disclosure, as one of the embodiments, the powder for inhalation further comprises an inhalable auxiliary material.

In the present disclosure, as one of the embodiments, the powder for inhalation comprises a combination of an inhalable auxiliary material and a lubricant.

In the present disclosure, as one of the embodiments, the inhalable auxiliary material includes lactose, mannitol, or an amino acid, or a combination of two or more thereof.

In the present disclosure, as one of the embodiments, the lubricant includes silicon dioxide, magnesium stearate, stearic acid, zinc stearate, calcium stearate, talc, sodium dodecyl sulfate, or a fatty acid ester. In the present disclosure, as one of the embodiments, the powder for inhalation comprises 10% of the drug-loaded particles, 89.8% of the inhalable auxiliary material, and 0.2% of the lubricant, based on the mass of the powder for inhalation.

In the present disclosure, as one of the embodiments, the powder for inhalation comprises 10% of the drug-loaded particles, 89.8% of lactose, and 0.2% of magnesium stearate, based on the mass of the powder for inhalation.

In the present disclosure, as one of the embodiments, the aerosol further comprises a propellant, and optionally further comprises a dispersant, a surfactant, or a combination thereof.

In the present disclosure, as one of the embodiments, the propellant includes tetrafluoroethane or heptafluoropropane.

In the present disclosure, as one of the embodiments, the dispersant includes PEG300.

In the present disclosure, as one of the embodiments, the surfactant is selected from polysorbate.

The present disclosure provides a preparation method for the pharmaceutical composition, and the method comprises the following steps:
(1) dissolving sivelestat or the salt thereof and the phospholipid in a solvent, or dissolving sivelestat or the salt thereof, the phospholipid, and the filler in a solvent, to obtain a drug solution;
(2) adjusting a pH of water for injection to 5.5 or less, and then mixing the water for injection with the drug solution to obtain a suspension; and
(3) spray-drying the suspension to obtain drug-loaded dry powder particles.

In the present disclosure, as one of the embodiments, the solvent in step (1) is selected from methanol, ethanol, diethyl ether, acetone, chloroform, n-hexane, dichloromethane, water, and a combination of two, three, or more thereof.

In the present disclosure, as one of the embodiments, the pH of water for injection is adjusted to 3.0-5.5 in step (2); as one of the embodiments, a pH adjuster for adjusting the pH is hydrochloric acid, citric acid, acetic acid, or an aqueous solution thereof.

In the present disclosure, as one of the embodiments, a mass ratio of the solvent to water for injection is 1:1 to 1:10.

In the present disclosure, as one of the embodiments, step (3) comprises: spray-drying the drug solution, with an inlet air temperature set to 70° C. to 110° C.

The present disclosure further provides a preparation method for the powder for inhalation, and the preparation method comprises the following step:
(3-1) spray-drying the suspension according to the claim to obtain drug-loaded dry powder particles, mixing the drug-loaded dry powder particles with the inhalable auxiliary material, optionally further adding the lubricant, and mixing to obtain the powder for inhalation.

In the present disclosure, as one of the embodiments, the inhalable auxiliary material includes lactose, mannitol, or an amino acid.

In the present disclosure, as one of the embodiments, the lubricant includes silicon dioxide, magnesium stearate, stearic acid, zinc stearate, calcium stearate, talc, sodium dodecyl sulfate, or a fatty acid ester.

The present disclosure further provides a preparation method for the aerosol of the pharmaceutical composition, and the preparation method comprises:
(3-2) spray-drying the suspension to obtain drug-loaded dry powder particles, mixing the drug-loaded dry powder particles with the propellant, optionally further adding the dispersant, the surfactant, or the combination thereof, and mixing to obtain the aerosol.

In the present disclosure, as one of the embodiments, the propellant includes tetrafluoroethane or heptafluoropropane; the dispersant includes PEG300; the surfactant is selected from polysorbate.

The present disclosure further provides a reconstituted formulation comprising dry powder particles prepared by the method of the present disclosure and water for injection, normal saline for injection, or aqueous glucose for injection.

In the present disclosure, as one of the embodiments, the dry powder particles of sivelestat or the salt thereof prepared by the present disclosure may be directly reconstituted with water for injection, normal saline for injection, or aqueous glucose for injection and then directly used in combination with another drug for a clinical patient.

In the present disclosure, sivelestat, which is poorly soluble in water, is used as a drug substance. The drug and a phospholipid (or further with a filler) are dissolved in a solvent, and the solution is then mixed with water for injection (pH 5.5 or less). Subsequently, the mixture is subjected to a spray drying process to form a powder. After particles of the powder are exposed to water, due to the amphiphilicity of the phospholipid and the hydrophobicity of the drug, the drug will be distributed between phospholipid bilayers or encapsulated by the phospholipid, spontaneously forming drug-loaded lipid particles.

Experiments demonstrated that the pulmonary drug exposure level of the pharmaceutical composition of the present disclosure was 1.8 times that of the micronized drug substance at 0.5 h, and was 11.0 times after 12 h.

The preparation process of the present disclosure is simple and easy for industrial production. After inhalation, the present disclosure achieves a high pulmonary exposure level and can release the drug in a sustained manner; the present disclosure is significantly superior to sivelestat particles without phospholipid coating. The present disclosure has a small amount of auxiliary materials, a simple preparation process, and a better sustained-release effect.

The advantages of the present disclosure are as follows:
1) The preparation process is simple and easy for industrial production.
2) The preparation process uses high-pressure homogenization, resulting in a relatively stable suspension.
3) Due to the sustained-release effect, the phospholipid-containing sivelestat suspension dosage form can treat respiratory diseases more effectively than other dosage forms.
4) After inhalation, the present disclosure achieves a high pulmonary exposure level and can release the drug in a sustained manner; the present disclosure is significantly superior to the inhalation of atomized solutions of sivelestat sodium powders for injection.

### BRIEF DESCRIPTION OF THE DRAWINGS

FIG. 1: a scanning electron micrograph of the sivelestat dry powder drug substance;

FIG. 2: a scanning electron micrograph of suspension particles of formula 1-1 before high-pressure homogenization;

FIG. 3: a scanning electron micrograph of suspension particles of formula 1-1 after high-pressure homogenization;

FIG. 4: a scanning electron micrograph of dry powder particles of formula 4-1;

FIG. 5: a scanning electron micrograph of dry powder particles of formula 5-1;

FIG. 6: a detection chromatogram of formula 5-1 on day 0;

FIG. 7: a detection chromatogram of formula 5-1 after 10 days of light exposure;

FIG. 8: a detection chromatogram of formula 5-1 after 10 days of standing at 40° C.;

FIG. 9: a detection chromatogram of formula 5-1 after 10 days of standing at 60° C.;

FIG. 10: a detection chromatogram of a sivelestat sodium suspension on day 0;

FIG. 11: a detection chromatogram of a sivelestat sodium suspension after 10 days of light exposure;

FIG. 12: a detection chromatogram of a sivelestat sodium suspension after 10 days of standing at 40° C.;

FIG. 13: a detection chromatogram of a sivelestat sodium suspension after 10 days of standing at 60° C.;

FIG. 14: scanning electron microscopy characterization of the micronized sivelestat sodium drug substance;

FIG. 15: scanning electron microscopy characterization of dry powder particles of formula 8-2; and

FIG. 16: scanning electron microscopy characterization of water-redispersed particles of a dry powder of formula 8-2.

### DETAILED DESCRIPTION OF THE INVENTION

The following examples are used to further illustrate the present disclosure, but do not limit the effective scope of the present disclosure in any way.

### Example 1: Test of Solubility of Sivelestat

Preparation of sivelestat: 40 g of sivelestat sodium was dissolved in 5 L of water for injection with stirring. The pH of the solution was then adjusted to 3.0 with 5% dilute hydrochloric acid, and sivelestat precipitated from the solution. The mixture was filtered, and the filter cake was washed and dried at 60° C. to give a sivelestat dry powder.

50 mL of water for injection (pH 6.8) was taken, and 5 mg of the sivelestat dry powder was added. The mixture was stirred at room temperature (25° C.) for 48 h and then filtered. The filtrate was analyzed to determine drug concentration, and the result was 0.0278 mg/mL.

The solubility of sivelestat sodium is 11.2 mg/mL at pH 6.8. Sivelestat is significantly less soluble in water than sivelestat sodium.

### Example 2: Screening for Lecithin Amount

The designed formulas are shown in Table 1 below:

TABLE 1

| | Formula | Formula | Formula | Formula | Formula | Formula | Formula | Formula | Formula | Formula | Formula |
|---|---|---|---|---|---|---|---|---|---|---|---|
| Composition | 1-1 (g) | 2-1 (g) | 3-1 (g) | 4-1 (g) | 5-1 (g) | 6-1 (g) | 7-1 (g) | 8-1 (g) | 9-1 (g) | 10-1 (g) | 11-1 (g) |
| Sivelestat | 1.0 | 1.0 | 1.0 | 1.0 | 1.0 | 1.0 | 1.0 | 1.0 | 1.0 | 0.1 | 10 |
| Egg yolk | 0.5 | 1.0 | 2.0 | 3.0 | 3.0 | 3.0 | 5.0 | 5.0 | 0.25 | 0.3 | 3.0 |
| lecithin | | | | | | | | | | | |
| Mannitol | 1.0 | 1.0 | 1.0 | 2.0 | 4.0 | 6.0 | 6.0 | 18.0 | 8.75 | / | / |
| Normal | / | / | / | / | / | / | / | / | / | 199.6 | 187.0 |
| saline | | | | | | | | | | | |

### Preparation of Sivelestat Suspension:

(1) Sivelestat and the phospholipid were ground and well mixed in a mortar to obtain a drug-phospholipid solid dispersion.
(2) 200 mL of normal saline was added to the solid dispersion, and the solid dispersion was uniformly dispersed with stirring to obtain a crude suspension.
(3) The crude suspension was homogenized 6 times under high pressure (700 bar) to obtain a suspension.

### Preparation of Sivelestat Dry Powder:

(4) Mannitol was added to the suspension obtained above in step (3). After dissolution was achieved by magnetically stirring the mixture at 1000 rpm, the solution was spray-dried at 110° C. to obtain the desired sivelestat dry powder.

After the spray drying process, it was observed that the powders of formula 3-1 and formula 4-1 (with a 50% lecithin proportion) exhibited relatively significant adhesion to the cyclone separator. As the lecithin proportion decreased, the powder adhesion to the cyclone separator gradually improved.

The suspensions of formulas 10-1 and 11-1 exhibited good stability; no significant sedimentation was observed at 3 h, and the sedimentation volume ratio was 1. Standing at room temperature for 30 days, both suspensions remained overall opaque. The suspension of formula 10-1 exhibited no sedimentation at the bottom. The suspension of formula 11-1 exhibited some sedimentation at the bottom due to the high drug concentration; however, the suspension could be homogenized with shaking.

To analyze the influence of different phospholipid/drug ratios on drug release, appropriate amounts of powders of formula 1-1, formula 2-1, formula 5-1, and formula 7-1 were uniformly dispersed in a certain amount of water for injection with stirring to prepare suspensions with a drug concentration of 5.0 mg/mL. 1.0 mL of each of the suspensions was placed in a dialysis tube (molecular weight cutoff: 300 kD) and then subjected to in vitro dissolution. The specific method is shown in Table 2-1 below:

TABLE 2-1

| Dissolution method | Paddle method, 50 rpm | |
|---|---|---|
| Dissolution medium | Phosphate-buffered saline, pH 6.8 | |
| Medium volume | 900 | mL |
| Medium temperature | 37 ± 0.5° | C. |
| Sampling volume | 10 | mL |

| | |
|---|---|
| Sampling time point | 0.5 h, 1 h, 2 h, 4 h, 8 h, 12 h |
| Replenish medium or not | Yes |
| Sample treatment and | After being passed through a |
| analysis method | 0.22 μm filter membrane, the samples |
| | were analyzed by high performance liquid |
| | chromatography. |

The results are shown in Table 2-2 below:

TABLE 2-2

| | 0.5 h(%) | 1 h(%) | 2 h(%) | 4 h(%) | 8 h(%) | 12 h(%) |
|---|---|---|---|---|---|---|
| Formula 1-1 | 44.1 | 74.5 | 92.7 | 100.4 | 100.8 | 99.8 |
| Formula 2-1 | 28.9 | 53.5 | 81.5 | 86.3 | 88.1 | 88.4 |
| Formula 5-1 | 14.7 | 26.9 | 43.2 | 58.1 | 65.0 | 65.9 |
| Formula 7-1 | 13.4 | 22.2 | 38.9 | 50.2 | 56.3 | 58.5 |

The dissolution results show that as the amount of the phospholipid increased, the proportion of the phospholipid coating the particles rose. Due to the hydrophobicity of the phospholipid, the drug dissolution rate slowed down. When the amount of the phospholipid was 0.5 times that of the drug (formula 1-1), the dissolution was complete after 4 h. When the amount of the phospholipid was 1 time that of the drug (formula 2-1), the dissolution amount remained substantially unchanged and did not exceed 90% after 4 h. When the amount of the phospholipid was 3 times that of the drug (formula 5-1), the dissolution amount remained substantially unchanged and did not exceed 70% after 8 h. When the amount of the phospholipid was 5 times that of the drug (formula 7-1), the dissolution rate slowed down and tended to be slower, and the dissolution amount did not exceed 60% after 12 h. As the amount of the phospholipid increased, release gradually slowed down, achieving a sustained-release effect. When the mass ratio of the phospholipid to the drug was greater than 1:1, the drug exhibited a sustained-release effect. When the mass ratio of the phospholipid to the drug was 3:1 to 5:1, the best effect was achieved.

### Example 3: Characterization of Particle Morphology

To further analyze the morphology of sivelestat particles, a small amount of the suspension of formula 1-1 before high-pressure homogenization and a small amount of the suspension of formula 1-1 after high-pressure homogenization were taken, dripped onto glass slides, and naturally dried. These samples, along with sivelestat dry powder particles, were subjected to scanning electron microscopy analysis. The results shown in FIGS. 1-3.

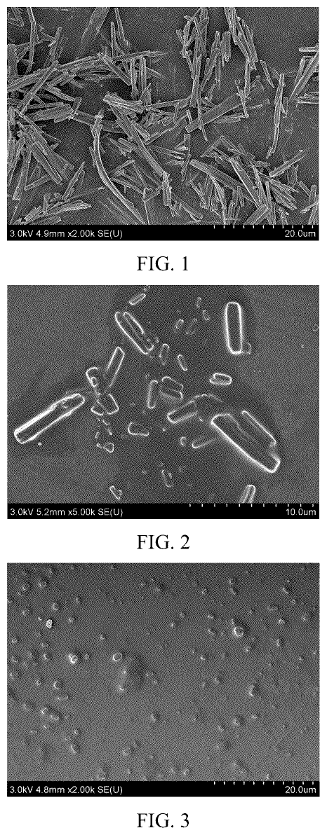

It can be seen from the scanning electron microscope results that sivelestat particles exhibited an elongated rod-like shape, with most particles ranging between 1-10 μm in length (FIG. 1). After manual grinding, the particles were broken into particles varying in length (FIG. 2). After a further high-pressure homogenization process, the particles all exhibited a short rod-like or sphere-like shape and were coated with a phospholipid layer (FIG. 3). According to analysis using a Nano ZS90 particle size analyzer, the average particle size was 730.7 nm.

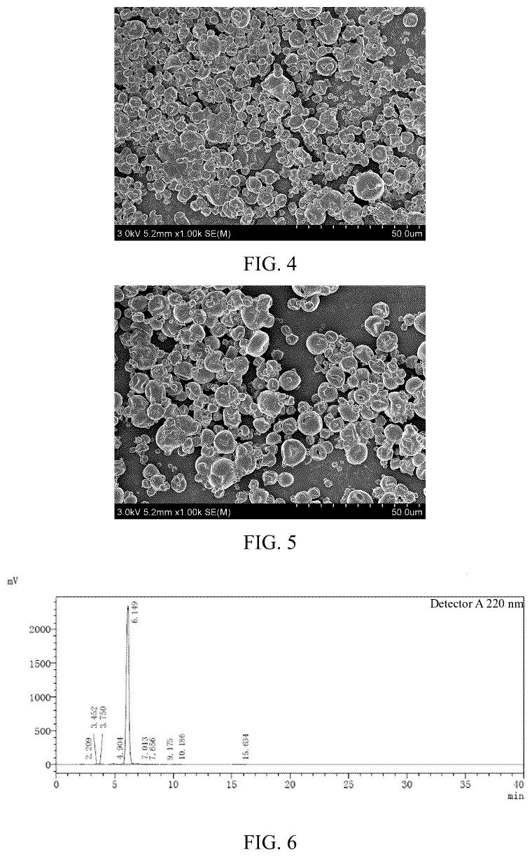

To further investigate the influence of different amounts of lecithin on dry powder particle morphology, the dry powder particles of formula 4-1 (with a 50% lecithin proportion) and formula 5-1 (with a 37.5% lecithin proportion) were subjected to scanning electron microscope analysis. The results are shown in FIGS. 4-5.

It can be seen from the results that after the spray drying treatment, the dry powder mainly consisted of sphere-like, wrinkled particles, and most primary particles were less than 5 μm in size. When the lecithin content was relatively high, powder particles easily adhered, leading to deformation. As the lecithin content decreased, the particle adhesion eased.

### Example 4: Comparison of Stability

To compare the stability of sivelestat and sivelestat sodium in water, 1.0 g of sivelestat sodium and 1.0 g of lecithin were homogeneously dispersed in 50 mL of water for injection, and the suspension was then aliquoted into vials (5 mL per vial). In addition, 1.0 g of the dry powder of formula 5-1 was dispersed in 50 mL of water for injection with shaking, and the suspension was then aliquoted into vials (5 mL per vial). The samples were placed in a 40° C. oven, a 60° C. oven, and a photostability tester, taken out after 10 days, and tested for related substances. The analysis method is shown in Table 3-1 below:

TABLE 3-1

| Chromatographic column | Waters XTerra RP18 3.9*150 mm 5 μm | |
|---|---|---|
| Column temperature | 25° | C. |
| Flow rate | 0.8 | mL/min |
| Detector | UV220 nm | |
| Mobile phase | A: 5.44 g of potassium dihydrogen | |
| | phosphate in 1000 mL of water, pH | |
| | adjusted to 3.5 with phosphoric acid | |
| | B: acetonitrile | |
| Ratio | A:B(55:45) | |
| Injection volume | 10 | μL |
| Run time | 40 | min |

Solvent: ethanol:water (1:1)

Test sample solution: A solution with a concentration of about 1 mg/mL was prepared.

Self-control solution: 1 mL of the test sample solution was added to a 100 mL volumetric flask, brought to volume with the solvent, and well shaken.

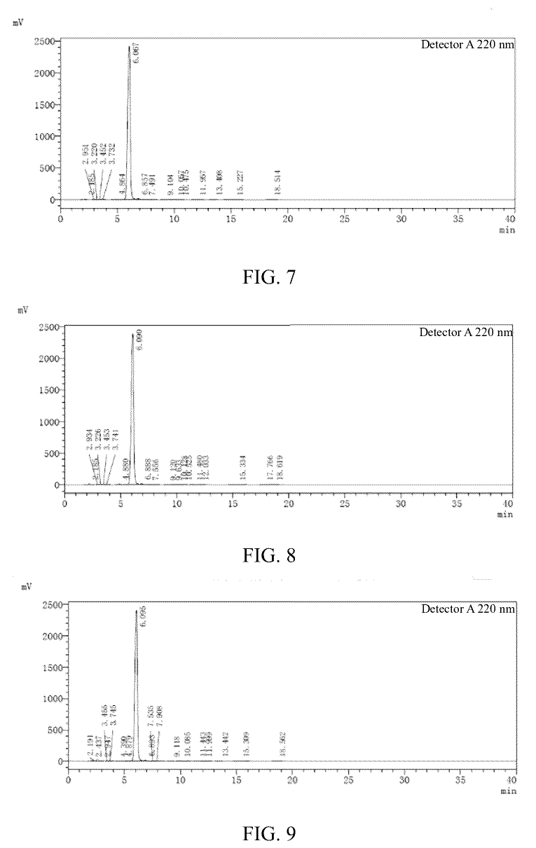

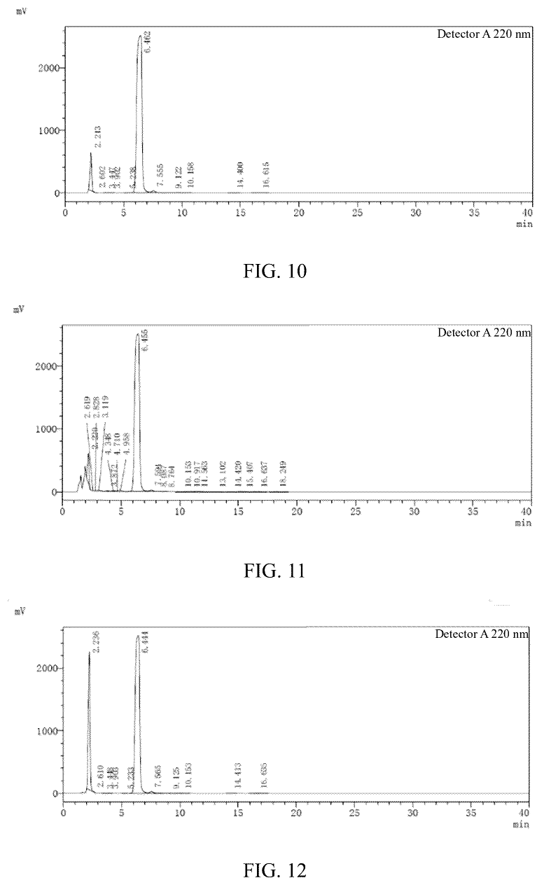

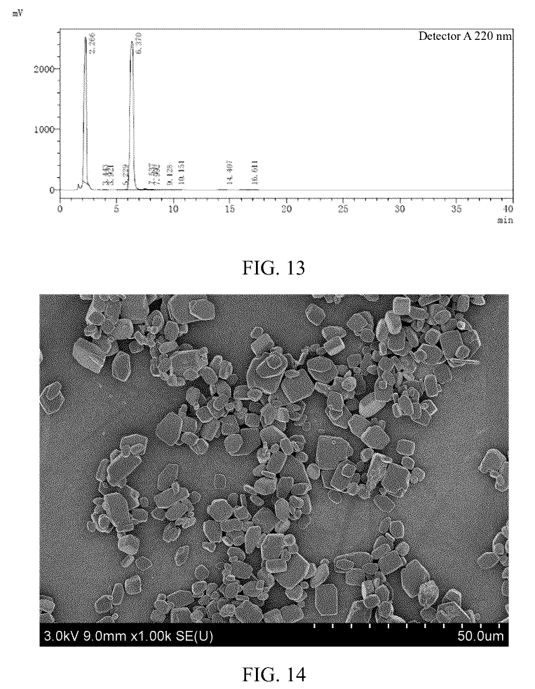

The results are shown in Table 3-2 below and FIGS. 6-13:

TABLE 3-2

| | | 40° C.- | 60° C.- | Light exposure |
|---|---|---|---|---|
| | 0 days | 10 days | 10 days | 10 days |
| Formula 5-1 | 0.63 | 0.74 | 0.84 | 0.62 |
| Sivelestat sodium | 0.76 | 20.05 | 47.86 | 4.19 |
| suspension | | | | |

Note: After 10 days of light exposure, the sivelestat sodium suspension was a brown suspension, and the other samples were all white to off-white suspensions.

It can be seen from the above results that sivelestat is significantly more stable in water than sivelestat sodium.

### Example 5: Determination of Atomization Performance

The mass median aerodynamic diameter (MMAD) is the most important key quality attribute for orally inhaled formulations. Particles with larger diameters (MMAD>10 μm) are typically filtered in the upper respiratory tract or nasopharynx due to inertial impaction; particles of 5-10 μm can reach the proximal end of the lower respiratory tract; particles of 1-5 μm are transported via airways to peripheral airways and alveoli; particles smaller than 0.5 μm are exhaled out of the body.

The aerodynamic particle size distribution of the dry powder of formula 5-1 and a water-redispersion suspension of the dry powder of formula 5-1 (0.5 mg/mL) was determined using a Next Generation Impactor (NGI). The dry powder was well mixed with lactose (twice the amount of the dry powder), and the mixture was then placed into capsules, with each capsule containing 1.0 mg of the drug. Another 16 mg of the dry powder was uniformly dispersed in 4 mL of water for injection with shaking to obtain a suspension with a drug concentration of 0.5 mg/mL, and 2.0 mL of the suspension was taken and atomized. Following standard pharmacopeial procedures, the specific results are shown in Table 4 below:

TABLE 4

| | | Dry powder |
|---|---|---|
| | | redispersed |
| | Dry powder | in water |
| MMAD(μm) | 3.6 | 3.3 |
| FPF₁(%) | 54.0 | 72.9 |
| FPF₂(%) | 46.8 | 32.6 |

FPF1: the percentage of the mass of particles with an aerodynamic particle size of less than 5 μm to the total drug amount delivered from the administration apparatus.
FPF2: the percentage of the mass of particles with an aerodynamic particle size of less than 5 μm to the labeled drug amount.

It can be seen from the results that both the dry powder and the redispersion suspension of the dry powder exhibited appropriate MMAD values, both their percentages of the mass of particles smaller than 5 μm to the total drug amount delivered from the administration apparatus were over 50%, and both their percentages of the mass of particles smaller than 5 μm to the labeled drug amount were over 30%, suggesting that they are suitable for being administered via inhalation and have relatively high deposition rates in the lungs.

### Example 6: Investigation of Exposure Level in Lung Tissue

A comparative study was conducted to investigate the pulmonary exposure levels of sivelestat suspension particles and a sivelestat sodium solution under different time conditions.

The samples and reagents are shown in Table 5-1 below:

TABLE 5-1

| Name | Manufacturer | Lot No. | Expiration date |
|---|---|---|---|
| Sivelestat reference standard | Beijing Jingzi Pharmaceutical | 20211102 | NA |
| | Technology Co., Ltd. | | |
| Phenytoin sodium control | National Institutes for Food and | 100210-202104 | NA |
| standard | Drug Control, PRC | | |
| Dimethyl sulfoxide | SIGMA-ALDRICH | STBH9909 | 2024 November |
| Acetonitrile | SIGMA-ALDRICH | K54636230241 | 2025 August |
| Citric acid | Beijing Mreda Technology Co., | M045481-500g | NA |
| | Ltd. | | |
| Potassium oxalate monohydrate | Beijing Mreda Technology Co., | M050145-50g | NA |
| | Ltd. | | |

| | |
|---|---|
| Deionized water | SIGMA-ALDRICH |
| SD rat blank lung tissue | Collected in-house |

### 6.1. Sample Preparation

Sivelestat suspension: 320 mg of the dry powder of formula 5-1 was uniformly dispersed in 40 mL of normal saline with shaking to obtain a 1.0 mg/mL suspension.

Sivelestat sodium solution: 2.0 g of the sivelestat sodium drug substance and 4.0 g of mannitol were uniformly suspended in 80.0 g of water for injection with stirring, and a 5% sodium hydroxide solution was then slowly added dropwise until the system became clear and transparent. Then the solution was brought to 100.0 g with water (pH 8.20). The solution was filtered using a 0.22 μm filter membrane, then aliquoted into vials (5.0 mL per vial), and freeze-dried to obtain a freeze-dried powder (the formula was the same as that of the commercially available powder for injection). Before use, water for injection was added to each vial of the freeze-dried powder to 5.0 mL, and the solution was then diluted with normal saline to 1.0 mg/mL to obtain a sivelestat sodium solution.

### 6.2. Experimental Animals

Experimental animals: 40 male SD rats weighing 220-240 g.

Experimental animal housing and management: The rats were housed in cages (2 per cage), given free access to maintenance feed and free access to water via drinking bottles.

Animal grouping and administration: The animals were randomized into 2 groups before administration, with 20 animals in each treatment group and 5 animals per time point, and the drugs were atomized and administered to the animals' airways.

Dose: 1 mg/kg.

After administration, the animals were anesthetized with isoflurane at 0.5 h, 1 h, 4 h, and 12 h. After anesthetization, blood was collected from the abdominal aorta, the animals were sacrificed, and lung tissue samples were collected.

### 6.3. Tissue Sample Treatment

Diluent 1: About 5 g of potassium oxalate monohydrate was weighed out and dissolved in an appropriate amount of ultrapure water. After thorough mixing, a 250 mg/mL aqueous potassium oxalate solution was obtained.
Diluent 2: About 10 g of citric acid was weighed out and dissolved in an appropriate amount of ultrapure water. After thorough mixing, a 500 mg/mL aqueous citric acid solution was obtained.
Preparation of homogenization buffer: 16 mL of dilution 1 and 8 mL of dilution 2 were added to 400 mL of normal saline to obtain a homogenization buffer containing 10 mg/mL citric acid and 10 mg/mL potassium oxalate.
After the lung tissue samples were taken, they were immediately weighed. The homogenization buffer (the mass-to-volume ratio of lung tissue to the homogenization buffer was 1:4 (g/mL)) was added, and the mixtures were immediately homogenized in an ice bath. The matrices were stored below −20° C.

### 6.4. Tissue Sample Analysis

50 μL of each lung tissue homogenate was taken, and 200 μL of acetonitrile (containing 20 ng·mL⁻¹ phenytoin sodium) was added. The mixtures were vortexed for 2 min and centrifuged at 45,000 rpm and 4° C. for 15 min. 10 μL of each supernatant was taken and diluted with 190 μL of acetonitrile/water (1:1), and LC-MS/MS mass spectrometry analysis was performed.

The pulmonary exposure level of the sivelestat suspension is shown in Table 5-2 below:

TABLE 5-2

| | Concentration (ng/g) | | | | | |
|---|---|---|---|---|---|---|
| Time (h) | 001 | 002 | 003 | 004 | 005 | Mean |
| 0.50 | 21040 | 13760 | 16160 | 15520 | 12440 | 15784 |
| 1 | 14880 | 11400 | 7360 | 8680 | 10800 | 10624 |
| 4 | 7200 | 6560 | 6920 | 6280 | 8970 | 7186 |
| 12 | 572 | 1552 | 900 | 1912 | 920 | 1171 |

The pulmonary exposure level of the sivelestat sodium solution is shown in Table 5-3 below:

TABLE 5-3

| | Concentration (ng/g) | | | | | |
|---|---|---|---|---|---|---|
| Time (h) | 001 | 002 | 003 | 004 | 005 | Mean |
| 0.50 | 5430 | 3780 | 1180 | 2450 | 3150 | 3198 |
| 1 | 1190 | 362 | 516 | 256 | 420 | 549 |
| 4 | 42.6 | 86.7 | 56.9 | 44.0 | 112 | 68.4 |
| 12 | 2.64 | 6.40 | 2.93 | 3.10 | 0.53 | 3.12 |

It can be seen from the results that after inhalation, the sivelestat suspension exhibited a high pulmonary exposure level and a good sustained-release effect. Its pulmonary drug exposure level was 4.9 times that of the sivelestat sodium solution at 0.5 h, was 19.4 times after 1 h, and was 375 times after 12 h.

In conclusion, these experiments of the present disclosure demonstrated that sivelestat has low solubility, a slow dissolution rate, and good stability. After being atomized and administered to rats' airways, the sivelestat suspension exhibited a high pulmonary exposure level and a good sustained-release effect; it was significantly superior to the sivelestat sodium solution.

### Example 7: Formula Preparation

(1) 2.0 g of the sivelestat or sivelestat sodium drug substance, a phospholipid, and a filler were placed into a beaker, and a solvent was added. The mixture was stirred for 10 min to dissolve the components, yielding a drug solution.
(2) The pH of water for injection was adjusted to 5.5 or less with 5% dilute hydrochloric acid, and the water for injection was then mixed with the drug solution under stirring conditions to obtain a suspension.
(3) The suspension was spray-dried (inlet air temperature: 110° C.) to obtain phospholipid drug-loaded dry powder particles.

The composition of different formulas is shown in Table 6 below:

TABLE 6

| | Formula a | Formula b | Formula c | Formula d | Formula e | Formula f |
|---|---|---|---|---|---|---|
| Composition | (g) | (g) | (g) | (g) | (g) | (g) |
| Sivelestat sodium | 2.0 | 2.0 | 2.0 | 2.0 | 2.0 | 2.0 |
| Egg yolk lecithin | 2.0 | 6.0 | 20 | / | / | / |
| Dipalmitoylphosphatidylethanolamine | / | / | / | 2.0 | 6.0 | 20 |
| Methanol | 30 | 90 | 300 | / | / | / |
| Methanol | / | | / | 108 | 324 | 1080 |
| Water for injection | 30 | 900 | 1500 | 108 | 700 | 3000 |
| pH of water for injection | 5.5 | 4.0 | 3.0 | 5.0 | 4.0 | 3.0 |

### Comparative Example 1

### Preparation of Particles Using the Same Formula and a Different Process

A certain amount of the sivelestat sodium drug substance and a phospholipid were placed into a beaker, and methanol was added. The mixture was stirred to dissolve the components, yielding a drug solution (the same as formula b).

The drug solution was spray-dried (inlet air temperature: 90° C.) to obtain phospholipid drug-loaded dry powder particles.

### In Vitro Dissolution Experiment of Different Formula Samples

Amounts of the powder of formula f described above, the powder of Comparative Example 1, and the micronized drug substance (average particle size: 2.4 μm), each equivalent to 100 mg of the drug, were weighed into dialysis bags (molecular weight cutoff: 300 kD), uniformly dispersed in 5.0 mL of a dissolution medium, and then subjected to in vitro dissolution. The dissolution method is shown in Table 7-1 below:

TABLE 7-1

| Total dissolution amount | 100 | mg |
|---|---|---|
| Dissolution method | Paddle method, 75 rpm | |
| Dissolution medium | Phosphate-buffered saline, pH 7.0 | |
| Medium volume | 900 | mL |
| Medium temperature | 37 ± 0.5° | C. |
| Sampling volume | 5.0 | mL |

| | |
|---|---|
| Sampling time point | 1 h, 6 h, and 12 h |
| Replenish medium or not | Yes |
| Sample treatment and | After being passed through a 0.22 μm |
| analysis method | filter membrane, the samples were |
| | analyzed by high performance liquid |
| | chromatography. |

(1) The results are shown in Table 7-2 below:

TABLE 7-2

| | 1 h(%) | 6 h(%) | 12 h(%) |
|---|---|---|---|
| Formula a | 20.1 | 48.6 | 82.7 |
| Formula b | 9.3 | 22.9 | 49.8 |
| Formula c | 8.0 | 21.1 | 44.9 |
| Formula d | 22.6 | 54.7 | 91.0 |
| Formula e | 19.4 | 36.2 | 70.5 |
| Formula f | 11.8 | 30.0 | 65.6 |
| Comparative Example 1 | 35.2 | 52.1 | 67.4 |
| Drug substance | 98.7 | 99.5 | 99.2 |

It can be seen from the dissolution results that the drug substance particles without phospholipid coating exhibited rapid release-its dissolution was complete at 1 h, and all phospholipid-containing formula particles exhibited incomplete dissolution at 1 h. At the same drug proportion, the use of egg yolk lecithin as the coating carrier resulted in a relatively slow drug release rate. As the proportion of the phospholipid carrier increased, the drug release rate slowed down. When the amount of egg yolk lecithin was not less than 3 times the drug amount, there was no significant difference in the drug release rate.

The formula of Comparative Example 1 was the same as formula b, but its preparation method was different from that of formula b, which caused the drug to be uniformly distributed in dry powder particles. As a result, part of the drug was present on the surface of the particles, leading to significant burst drug release; therefore, it failed to achieve a sustained-release effect.

The types and mass of the phospholipids, fillers, and solvents used in the experiment are shown in Table 7-3.

TABLE 7-3

| | Formula | Formula | Formula | Formula | Formula | Formula | Formula | Formula | Formula |
|---|---|---|---|---|---|---|---|---|---|
| Composition | 1-2 (g) | 2-2 (g) | 3-2 (g) | 4-2 (g) | 5-2 (g) | 6-2 (g) | 7-2 (g) | 8-2 (g) | 9-2 (g) |
| Sivelestat | 2.0 | 2.0 | 2.0 | 2.0 | 2.0 | / | / | 1 | / |
| Sivelestat sodium | / | / | / | / | / | 2.0 | 2.0 | 2.0 | 1.0 |
| Egg yolk lecithin | 4.0 | 4.0 | 4.0 | / | / | 0.5 | 2.0 | 6.0 | 5.0 |
| Soybean phospholipid | / | / | / | 4.0 | / | / | / | / | / |
| Dioleoylphosphatidylcholine | / | / | / | / | 4.0 | / | / | / | / |
| Povidone K30 | / | / | 8.0 | 8.0 | 8.0 | 4.0 | 4.0 | 12 | 10 |
| Sorbitol | 1.0 | 4.0 | / | / | / | / | / | / | / |
| Methanol | / | / | / | 300 | 300 | 300 | 300 | 300 | 300 |
| 75% ethanol | 300 | 300 | 300 | / | / | / | / | / | / |

In the case that the amount of egg yolk lecithin and the amount of the drug were fixed, as the amount of the filler increased, the powder formation capacity of the particles of formulas 1-2 to 3-2 improved, and their yields gradually increased. Due to the relatively large proportion of egg yolk lecithin, which is waxy, almost all particles of formula 1-2 tightly adhered to the walls of the atomization chamber and the cyclone separator, such that the particles could not be effectively collected. In contrast, the particles of formulas 2-2 and 3-2 could be collected, with yields of about 39% and about 82%, respectively. When the content of the filler was more than 15%, the powder formation capacity of the dry powder particles effectively improved; the best effect was achieved when the content was 50% to 70%.

### Experimental Example 1: In Vitro Dissolution Experiment of Different Formula Samples

Amounts of the powders of formulas 3-2 to 9-2 in Example 7, each equivalent to 10 mg of the drug, were weighed into dialysis bags (molecular weight cutoff: 300 kD), uniformly dispersed in 5.0 mL of a dissolution medium, and then subjected to in vitro dissolution. For better dissolution comparison, 50 mg of the sivelestat drug substance was dispersed in 25 mL of Tween 80 solution, and 5.0 mL of the suspension was used as a comparative example for the experiment.

The dissolution method is shown in Table 8-1 below:

TABLE 8-1

| Dissolution method | Paddle method, 75 rpm | |
|---|---|---|
| Dissolution medium | Phosphate-buffered saline, pH 6.8 | |
| Medium volume | 900 | mL |
| Medium temperature | 37 ± 0.5° | C. |
| Sampling volume | 5.0 | mL |

| | |
|---|---|
| Sampling time point | 0.5 h, 1 h, 2 h, 4 h, and 6 h |
| Replenish medium or not | Yes |
| Sample treatment and analysis | After being passed through a 0.22 μm |
| method | filter membrane, the samples were |
| | analyzed by high performance liquid |
| | chromatography. |

The results are shown in Table 8-2 below:

TABLE 8-2

| | 0.5 h | 1 h(%) | 2 h(%) | 4 h(%) | 6 h(%) |
|---|---|---|---|---|---|
| Formula 3-2 | 27.5 | 47.1 | 65.6 | 89.7 | 101.0 |
| Formula 4-2 | 26.6 | 48.5 | 68.2 | 91.0 | 99.8 |
| Formula 5-2 | 35.9 | 56.4 | 77.4 | 100.9 | 100.5 |
| Formula 6-2 | 52.4 | 75.2 | 99.6 | 99.7 | 99.4 |
| Formula 7-2 | 41.6 | 58.3 | 80.2 | 100.3 | 100.8 |
| Formula 8-2 | 22.0 | 39.7 | 56.0 | 90.6 | 99.3 |
| Formula 9-2 | 19.1 | 35.8 | 51.9 | 90.8 | 98.9 |
| Comparative example | 70.6 | 100.5 | 100.2 | 99.9 | 99.5 |

It can be seen from the dissolution results that the most rapid drug dissolution occurred when there was no phospholipid coating. The sustained-release effects of egg yolk lecithin and soybean phospholipid on the drug were similar (formulas 3-2 and 4-2), while the sample containing dioleoylphosphatidylcholine (formula 5-2) exhibited relatively rapid dissolution. Comparisons of these three phospholipids show that the use of egg yolk lecithin and soybean phospholipid resulted in better sustained drug release effects. As the phospholipid/drug ratio increased, the drug release rate slowed down. When the phospholipid/drug ratio was 3 (formula 8-2) and 5 (formula 9-2), there was no significant difference in drug release. When the mass of the phospholipid was 3 times that of the drug, the phospholipid drug-loaded particles effectively achieved a sustained-release effect; the best effect was achieved when the mass ratio of the phospholipid to the drug was 3:1 to 6:1.

### Experimental Example 2: Characterization of Particle Morphology

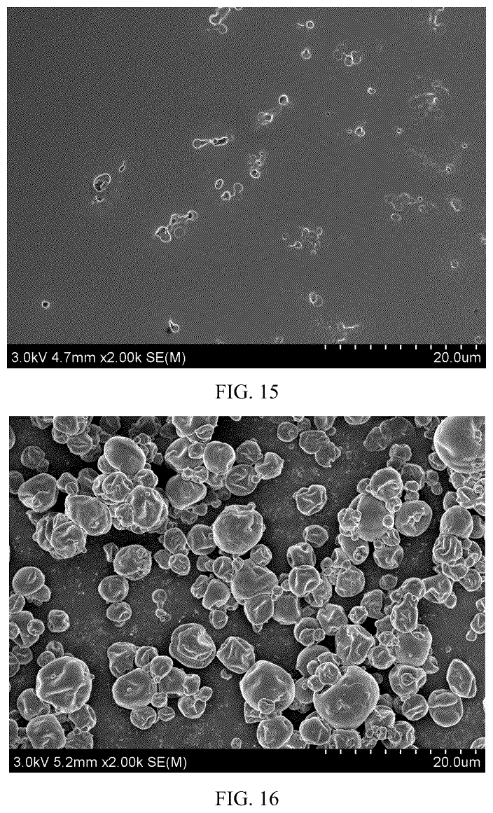

The micronized sivelestat sodium drug substance and the dry powder of formula 8-2 were characterized by scanning electron microscopy. Their results are shown in FIG. 14 and FIG. 15, respectively. In addition, to determine the morphology of particles after redispersion of the spray-drying powder in water, a small amount of the dry powder of formula 8-2 was uniformly dispersed in 2.0 mL of water with shaking. An appropriate amount of the suspension was then dripped onto a glass slide and air-dried at room temperature. After gold sputtering, scanning electron microscopy characterization was performed. The result is shown in FIG. 16.

It can be seen from the result that particles of the micronized sivelestat sodium drug substance exhibited an irregular, flaky shape, with most particles less than 5.0 μm in width. The particles after spray drying were sphere-like dented particles varying in size, with most particles ranging between 1-5 μm in size. The particles after redispersion of the dry powder in water exhibited a uniform, sphere-like shape, and their particle size was less than 2.0 μm.

### Experimental Example 3: Investigation of Exposure Level in Lung Tissue

The samples and reagents are shown in Table 9-1:

TABLE 9-1

| | | | Expiration |
|---|---|---|---|
| Name | Manufacturer | Lot No. | date |
| Sivelestat reference standard | Beijing Jingzi Pharmaceutical | 20211102 | NA |
| | Technology Co., Ltd. | | |
| Phenytoin sodium control | National Institutes for Food and | 100210-202104 | NA |
| standard | Drug Control, PRC | | |
| Dimethyl sulfoxide | SIGMA-ALDRICH | STBH9909 | 2024 November |
| Acetonitrile | SIGMA-ALDRICH | K54636230241 | 2025 August |
| Citric acid | Beijing Mreda Technology Co., Ltd. | M045481-500g | NA |
| Potassium oxalate | Beijing Mreda Technology Co., Ltd. | M050145-50g | NA |
| monohydrate | | | |

| | |
|---|---|
| Deionized water | SIGMA-ALDRICH |
| SD rat blank lung tissue | Collected in-house |

### 3.1. Sample Preparation

To better investigate the exposure level in rat lung tissue, the powder of formula 8-2 was ultrasonically and uniformly dispersed in water for injection to obtain a sustained-release particle suspension with a drug concentration of 10 mg/mL. The micronized sivelestat sodium drug substance was uniformly dispersed in a 0.1% aqueous Tween 80 solution with stirring to obtain a drug substance suspension with the same drug concentration, i.e., 10 mg/mL.

### 3.2. Experimental Animals

Experimental animals: 30 male SD rats weighing 220-240 g.

Experimental animal housing and management: The rats were housed in cages (3 per cage), given free access to maintenance feed and free access to water via drinking bottles.

### Animal Grouping and Administration

Before administration, the animals were randomized into 2 groups, with 15 animals in each treatment group and 5 animals per time point.

Dose: 8 mg/kg.

After administration, the animals were anesthetized with isoflurane at 0.5 h, 4 h, and 12 h. After anesthetization, blood was collected from the abdominal aorta, the animals were sacrificed, and lung tissue samples were collected.

### 3.3. Tissue Sample Treatment

Diluent 1: About 5 g of potassium oxalate monohydrate was weighed out and dissolved in an appropriate amount of ultrapure water. After thorough mixing, a 250 mg/mL aqueous potassium oxalate solution was obtained.
Diluent 2: About 10 g of citric acid was weighed out and dissolved in an appropriate amount of ultrapure water. After thorough mixing, a 500 mg/mL aqueous citric acid solution was obtained.
Preparation of homogenization buffer: 16 mL of dilution 1 and 8 mL of dilution 2 were added to 400 mL of normal saline to obtain a homogenization buffer containing 10 mg/mL citric acid and 10 mg/mL potassium oxalate.

After the lung tissue samples were taken, they were immediately weighed. The homogenization buffer (the mass-to-volume ratio of lung tissue to the homogenization buffer was 1:4 (g/mL)) was added, and the mixtures were immediately homogenized in an ice bath. The matrices were stored below −20° C.

### 3.4. Tissue Sample Analysis

50 μL of each lung tissue homogenate was taken, and 200 μL of acetonitrile (containing 20 ng·mL⁻¹ phenytoin sodium) was added. The mixtures were vortexed for 2 min and centrifuged at 45,000 rpm and 4° C. for 15 min. 10 μL of each supernatant was taken and diluted with 190 μL of acetonitrile/water (1:1), and LC-MS/MS mass spectrometry analysis was performed.

The results for the micronized drug substance are shown in Table 9-2 below:

TABLE 9-2

| Time | | 01(ng/g) | 02(ng/g) | 03(ng/g) | 04(ng/g) | 05(ng/g) | Mean (ng/g) |
|---|---|---|---|---|---|---|---|
| 0.5 | h | 127200 | 70800 | 118800 | 49200 | 71200 | 87440 |
| 4 | h | 14880 | 11400 | 7360 | 8680 | 10800 | 10624 |
| 12 | h | 11520 | 4040 | 312 | 4640 | 2960 | 4694 |

The results for the sustained-release particles are shown in Table 9-3 below:

TABLE 9-3

| Time | | 01(ng/g) | 02(ng/g) | 03(ng/g) | 04(ng/g) | 05(ng/g) | Mean (ng/g) |
|---|---|---|---|---|---|---|---|
| 0.5 | h | 64000 | 157600 | 100800 | 68400 | 385200 | 155200 |
| 4 | h | 100000 | 146400 | 132000 | 50800 | 153200 | 116480 |
| 12 | h | 92800 | 64400 | 32960 | 23520 | 45200 | 51776 |

It can be seen from the results that compared to the micronized drug substance particles, the phospholipid drug-loaded sustained-release particles, after atomization and inhalation, exhibited a high pulmonary exposure level: their pulmonary drug exposure level was 1.8 times that of the drug substance at 0.5 h, and was 11.0 times after 12 h; and a good sustained-release effect: the pulmonary drug exposure level of the micronized drug substance changed from 87,440 ng/g to 10,624 ng/g (from 0.5 h to 4 h), which was an approximately 8-fold reduction, while the pulmonary drug exposure level of the sustained-release particles of the present disclosure changed from 155,200 ng/g to 116,480 ng/g (from 0.5 h to 4 h), showing a significant sustained-release effect.

### Experimental Example 4: Preparation of Powder for Inhalation

According to formula 2-2, drug-loaded dry powder particles (10.0 g, containing 2.0 g of the drug) were prepared for later use.

45.0 g of lactose was weighed into a material bag, then 0.2 g of magnesium stearate was added, and then 44.8 g of lactose was added. After sealing, they were mixed in a hopper blender at 15 rpm for 20 min. About half of the lactose/magnesium stearate mixture was placed in a material bag, 10.0 g of the drug-loaded dry powder was added, and the remaining portion of the lactose/magnesium stearate mixture was then added. After sealing, they were mixed in a hopper blender at 15 rpm for 20 min. Finally, the well-mixed powder was placed into gelatin capsules, with each capsule filled with 25 mg of the powder (containing 0.5 mg of the drug).

The aerodynamic particle size distribution of the powder for inhalation was determined using a New Generation Impactor (NGI). The results showed that its mass median aerodynamic diameter was 3.2 μm, and its percentage of the mass of particles with an aerodynamic particle size of less than 5 μm to the labeled drug amount was 43.6%. Since particles of 1-5 μm can be transported via airways to peripheral airways and alveoli, this indicates that after the drug-loaded dry powder particles are prepared into an aerosol, they have a relatively high deposition rate in the lungs and thus can well exert their therapeutic effect.

1. A sustained-release sivelestat-containing pharmaceutical composition, comprising sivelestat or a salt thereof and a phospholipid.

2. The pharmaceutical composition according to claim 1, wherein sivelestat and the phospholipid form a solid dispersion.

3. The pharmaceutical composition according to claim 1, wherein a mass ratio of sivelestat to the phospholipid is 1:1 to 1:6, preferably 1:3 to 1:5.

4. The pharmaceutical composition according to claim 1, wherein the pharmaceutical composition comprises sivelestat, the phospholipid, and an aqueous solution and does not comprise an organic solvent.

5. The pharmaceutical composition according to claim 3, wherein in the composition, sivelestat accounts for 0.05-5%, preferably 0.1-3.0%, by mass of the composition, and the phospholipid accounts for 0.15-1.5%, preferably 0.3-1.0%, by mass of the composition.

6. The pharmaceutical composition according to claim 5, wherein the pharmaceutical composition comprises sivelestat, the phospholipid, and a drying protectant and does not comprise an organic solvent.

7. The pharmaceutical composition according to claim 6, wherein in the composition, sivelestat accounts for 5-40%, preferably 10-30%, by mass of the composition, and the phospholipid accounts for 2.5-50%, preferably 5-42%, more preferably 10-37.5%, and further preferably 15-35%, by mass of the composition.

8. The pharmaceutical composition according to claim 7, wherein the phospholipid is selected from egg yolk lecithin, soybean phospholipid, a synthetic phospholipid, and a combination of two or more thereof, preferably, the phospholipid is egg yolk lecithin.

9. The pharmaceutical composition according to claim 6, wherein the drying protectant accounts for 25-75%, preferably 40-60%.

10. The pharmaceutical composition according to claim 9, wherein the drying protectant is one of or a combination of two or more of fructose, xylitol, maltitol, erythritol, povidone, polyethylene glycol, hydroxypropyl methylcellulose, hydroxypropyl cellulose, poloxamer, lactose, glucose, sucrose, trehalose, mannitol, and sorbitol, preferably one of or a combination of two or more of lactose, glucose, sucrose, trehalose, mannitol, and sorbitol, and further preferably mannitol.

11. A formulation comprising the pharmaceutical composition according to claim 1, wherein a dosage form is a freeze-dried powder for injection, a powder for inhalation, an aerosol, or a suspension.

12. A method for preparing the pharmaceutical composition according to claim 1, comprising:
(i) directly grinding sivelestat and the phospholipid to obtain a drug-phospholipid solid dispersion; and
(ii) adding water for injection to the solid dispersion, and mixing well (preferably homogenizing under increased pressure) to obtain a suspension.

13. The method according to claim 12, further comprising:
(iii) spray-drying the suspension obtained in step (ii) or adding a drying protectant to the suspension, mixing well, and then spray-drying to obtain dry powder particles.

14. The method according to claim 12, wherein the method comprises in step (ii): the homogenizing under increased pressure refers to performing homogenization 1-10 times under a pressure of 200-1000 bar; preferably, homogenization is performed 6 times under a pressure of 700 bar.

15. The method according to claim 13, wherein the method comprises in step (iii): spray drying is performed at a temperature of 90-110° C.

16. The pharmaceutical composition according to claim 1, wherein in the pharmaceutical composition, the sivelestat or the salt thereof and the phospholipid form phospholipid particles loaded with sivelestat or the salt thereof.

17. The pharmaceutical composition according to claim 1, wherein in the pharmaceutical composition, the phospholipid accounts for 10-60%, preferably 20-50%, by mass of the pharmaceutical composition.

18. The pharmaceutical composition according to claim 1, wherein in the pharmaceutical composition, a mass ratio of the phospholipid to sivelestat or the salt thereof is 1:1 to 10:1, preferably 2:1 to 8:1, and further preferably 3:1 to 6:1, based on the mass of the pharmaceutical composition.

19. The pharmaceutical composition according to claim 1, wherein the phospholipid is selected from egg yolk lecithin, soybean phospholipid, a synthetic phospholipid, and a combination of two or more thereof, preferably, the phospholipid is egg yolk lecithin or soybean phospholipid; further preferably, the phospholipid is egg yolk lecithin.

20. The pharmaceutical composition according to claim 1, wherein in the pharmaceutical composition, sivelestat or the salt thereof accounts for 1-50%, preferably 5-40%, and more preferably 10-30%, by mass of the pharmaceutical composition.

21. The pharmaceutical composition according to claim 1, wherein the salt of sivelestat includes sivelestat sodium.

22. The pharmaceutical composition according to claim 1, further comprising a filler, wherein the filler accounts for 15-90%, preferably 35-80%, and more preferably 50-70%, by mass of the pharmaceutical composition.

23. The pharmaceutical composition according to claim 22, wherein the filler includes sorbitol, mannitol, lactose, sucrose, glucose, an amino acid, povidone, copovidone, poloxamer, polyethylene glycol, or a combination of two or more thereof, preferably, the filler is sorbitol, mannitol, lactose, an amino acid, or a combination of two or more thereof.

24. The pharmaceutical composition according to claim 1, wherein the pharmaceutical composition has a pH of 3.0-5.5.

25. The pharmaceutical composition according to claim 24, further comprising an adjuster for the pH, wherein the adjuster for the pH includes hydrochloric acid, citric acid, acetic acid, or an aqueous solution thereof, preferably, the hydrochloric acid solution is a 5% dilute hydrochloric acid solution.

26. A formulation according to claim 16, wherein the formulation includes a freeze-dried powder for injection, a suspension, a powder for inhalation, or an aerosol.

27. The formulation according to claim 26, wherein the powder for inhalation further comprises an inhalable auxiliary material or a combination of an inhalable auxiliary material and a lubricant;
the inhalable auxiliary material includes lactose, mannitol, or an amino acid, or a combination of two or more thereof, and
the lubricant includes silicon dioxide, magnesium stearate, stearic acid, zinc stearate, calcium stearate, talc, sodium dodecyl sulfate, or a fatty acid ester.

28. The formulation according to claim 27, wherein the powder for inhalation comprises 10% of the drug-loaded particles, 89.8% of the inhalable auxiliary material, and 0.2% of the lubricant, based on the mass of the powder for inhalation.

29. The formulation according to claim 28, wherein the powder for inhalation comprises 10% of the drug-loaded particles, 89.8% of lactose, and 0.2% of magnesium stearate, based on the mass of the powder for inhalation.

30. The formulation according to claim 26, wherein the aerosol further comprises a propellant, and optionally further comprises a dispersant, a surfactant, or a combination thereof.

31. The formulation according to claim 30, wherein the propellant includes tetrafluoroethane or heptafluoropropane; the dispersant includes PEG300; the surfactant is selected from polysorbate.

32. A method for preparing the pharmaceutical composition according to claim 16, comprising:
(i) dissolving sivelestat or the salt thereof and the phospholipid in a solvent, or dissolving sivelestat or the salt thereof, the phospholipid, and the filler in a solvent, to obtain a drug solution; and
(ii) adjusting a pH of water for injection to 5.5 or less, and then mixing the water for injection with the drug solution to obtain a suspension; and optionally further comprising:
(iii) spray-drying the suspension to obtain drug-loaded dry powder particles.

33. The method according to claim 32, wherein the solvent in step (i) is selected from methanol, ethanol, diethyl ether, acetone, chloroform, n-hexane, dichloromethane, water, and a combination of two, three, or more thereof.

34. The method according to claim 32, wherein the pH of water for injection is adjusted to 3.0-5.5 in step (ii); preferably, with a pH adjuster for adjusting the pH is hydrochloric acid, citric acid, acetic acid, or an aqueous solution thereof.

35. The method according to claim 32, wherein a ratio of the drug solution to water is 1:1 to 1:10.

36. The method according to claim 32, wherein step (iii) comprises: spray-drying the drug solution, with an inlet air temperature set to 70° C. to 110° C.

37. A method for preparing the powder for inhalation according to claim 27, comprising:
(i) dissolving sivelestat or the salt thereof and the phospholipid in a solvent, or dissolving sivelestat or the salt thereof, the phospholipid, and the filler in a solvent, to obtain a drug solution; and
(ii) adjusting a pH of water for injection to 5.5 or less, and then mixing the water for injection with the drug solution to obtain a suspension; and optionally further comprising:
(iii) spray-drying the suspension to obtain drug-loaded dry powder particles;
(iii-a) mixing the drug-loaded dry powder particles obtained in (iii) with the inhalable auxiliary material, optionally further adding the lubricant, and mixing to obtain the powder for inhalation.

38. The method according to claim 37, wherein the inhalable auxiliary material includes lactose, mannitol, or an amino acid.

39. The method according to claim 37, wherein the lubricant includes silicon dioxide, magnesium stearate, stearic acid, zinc stearate, calcium stearate, talc, sodium dodecyl sulfate, or a fatty acid ester.

40. A method for preparing the aerosol according to claim 30, comprising:
(i) dissolving sivelestat or the salt thereof and the phospholipid in a solvent, or dissolving sivelestat or the salt thereof, the phospholipid, and the filler in a solvent, to obtain a drug solution; and
(ii) adjusting a pH of water for injection to 5.5 or less, and then mixing the water for injection with the drug solution to obtain a suspension; and optionally further comprising:
(iii) spray-drying the suspension to obtain drug-loaded dry powder particles;
(iii-b) mixing the drug-loaded dry powder particles obtained in (3) with the propellant, optionally further adding the dispersant, the surfactant, or the combination thereof, and mixing to obtain the aerosol.

41. The method according to claim 40, wherein the propellant includes tetrafluoroethane or heptafluoropropane; the dispersant includes PEG300; and the surfactant is polysorbate.

42. A reconstituted formulation, comprising dry powder particles prepared by the method according to claim 12 and water for injection, normal saline for injection, or aqueous glucose for injection.

43. A method for treating bronchiectasis, acute lung injury, or acute respiratory distress syndrome, comprising administering the pharmaceutical composition according to claim 1, to subject in need thereof.
